(19) United States
(12) Patent Application Publication
(10) Pub. No.: US 2017/0050030 A1
(43) Pub. Date: Feb. 23, 2017

(54) ADAPTER FOR ATTACHING AN ELECTROMEDICAL SIGNAL TRANSMITTER TO AN IMPLANTABLE ELECTROMEDICAL DEVICE

(71) Applicant: Osypka AG, Rheinfelden-Baden (DE)

(72) Inventors: Thorsten Gottsche, Rheinfelden (DE); Maik Grafe, Rheinfelden (DE); Francois Alesch, Kirchbach (AT)

(73) Assignee: Osypka AG, Rheinfelden-Baden (DE)

(21) Appl. No.: 15/119,041

(22) PCT Filed: Mar. 18, 2015
(86) PCT No.: PCT/EP2015/000598

§ 371 Date: Aug. 15, 2016

(30) Foreign Application Priority Data
Mar. 18, 2014 (DE) 10 2014 003 843.2

(51) Int. Cl. A61N 1/375; H01R 13/73

(52) U.S. Cl. CPC A61N 1/3752; H01R 13/73; A61N 1/362

(57) ABSTRACT

The adapter (1) implantable in a human body has the first electrical terminal (2) with at least one electrical contact (3) for attaching to an implantable electromedical device (4) and the second electrical terminal (5) with at least one electrical contact (6) for attaching to the implantable electromedical signal transmitter (7), in particular the stimulation electrode (26) or the extension (25) or a sensing electrode. The first electrical terminal (2) and the second electrical terminal (5) of the adapter (1) are electrically connected to each other in order to transmit electromedical signals. The adapter (1) includes the housing (8), on which the first electrical terminal (2) and the second electrical terminal (5) are formed. On the housing (8), the recess (9) is formed which is designed to receive and engage around at least a part of the implantable electromedical device (4).

### BACKGROUND

The invention relates to an adapter implantable in a body, having a first electrical terminal with at least one electrical contact for attaching an implantable electromedical device, and having a second electrical terminal with at least one electrical contact for attaching an implantable electromedical signal transmitter which, in the implanted position of use, is designed to transmit electromedical signals between the implantable electromedical device and the body, wherein the first electrical terminal and the second electrical terminal of the adapter are electrically connected to each other in order to transmit electromedical signals.

Moreover, the invention also relates to such an adapter with an implantable electromedical signal transmitter attached to the adapter in the position of use, to such an adapter with an implantable electromedical device attached to the adapter in the position of use, and to an aforementioned adapter with an implantable electromedical device attached to its first electrical terminal in the position of use and with an implantable electromedical signal transmitter attached to its second electrical terminal in the position of use.

Implantable electromedical devices, and implantable electromedical signal transmitters too, are available on the market from many different manufacturers. To achieve the best possible result when using such devices and signal transmitters, it may be expedient to combine an electromedical device from one manufacturer with an electromedical signal transmitter from another manufacturer. Since the manufacturers on the market have oftentimes defined different and therefore incompatible interface standards for attachment of electromedical signal transmitters to electromedical devices, attachment of an electromedical signal transmitter to an electromedical device from another manufacturer is in such cases only possible with the aid of an adapter as defined at the outset.

The adapters hitherto available on the market and allowing the different interface standards to be bridged consist, for example, of a cable which, at one of its two ends, has a terminal that fits the electromedical signal transmitter and, at the other end, has a terminal that fits the respective electromedical device.

However, when using these adapters, also designated as cable adapters, it may be necessary to widen the tissue pocket usually created in the body for accommodating this adapter, so as to be able to receive the electromedical device that is to be implanted. Moreover, after it has been placed in the tissue pocket, the adapter cable is then however arranged more or less loosely in this tissue pocket, which can result not only in medical problems but also cosmetic problems, since the adapter cable is able to move freely in the tissue pocket and can lead to bumps or bulges visible from the outside.

### SUMMARY

The object of the invention is therefore to make available an adapter which is as defined at the outset and with which it is possible to avoid these disadvantages.

In the case of the adapter defined at the outset and implantable in a body, this object is achieved by one or more features of the invention and in particular by the fact that the adapter has a housing, on which the first electrical terminal and the second electrical terminal are formed, and that the housing has a recess, which is designed to receive and engage around at least part of the implantable electromedical device. It is thus possible that the adapter, in the position of use, engages around at least part of the implantable electromedical device and is thus connected to the electromedical device and forms a compact unit with the electromedical device. In the position of use, the adapter can engage around or over only a defined part of the electromedical device, for example the head part or header thereof, or else can enclose the adapter over greater parts.

When using an adapter according to the invention, the engagement of the adapter around the impulse transmitter can have the advantage that the tissue pocket, into which the implantable device is fitted together with the adapter in the position of use, does not have to be widened or at least only has to be widened to a lesser extent than in the case of the previously known adapters. Moreover, the adapter can be secured on the electromedical device by receiving the electromedical device in its housing-side recess, by which it is possible to avoid medical or functional problems, caused by uncontrolled and unwanted shifting of the adapter in the tissue pocket, and often associated cosmetic problems.

To be able to secure an electromedical device in the recess of the adapter, it may be expedient if the adapter has at least one securing means, in particular for releasable and/or form-fit and/or force-fit engagement of the electromedical device inserted into the recess in the position of use. Particularly if the securing means is designed for releasably securing the electromedical device inserted in the recess, it is possible to remove the electromedical device from the recess of the adapter for maintenance purposes and/or for replacement, without thereby damaging the implanted adapter or having to remove it from its implanted position of use.

It may be particularly advantageous if the first electrical terminal for the implantable electromedical device is formed in the interior of the recess of the housing, and the second electrical terminal for the implantable electromedical signal transmitter is preferably formed on an externally accessible side or outer face of the housing. When the second electrical terminal for the implantable electromedical signal transmitter is arranged on the outside of the housing of the adapter, attachment of the signal transmitter to the adapter can be easily carried out, for example, even during a medical intervention. An arrangement of the first electrical terminal for the electromedical device in the interior of the recess of the housing of the impulse transmitter may be advantageous in order to protect this terminal from external influences, as a result of which it is then possible to obtain fault-free operation of the implantable electromedical device.

At least one wall of the housing delimiting the recess can be designed for preferably leak-tight closure of a gap present between the wall and the implantable electromedical device inserted into the recess. Moreover, it is possible that the wall has a means for preferably leak-tight closure, in particular a sealing lip. In this way, the implantable electromedical device inserted into the recess, and in particular the first terminal for the device, can be better protected against admission of liquid and thus against defects or failure.

The insertion of the electromedical device into the recess of the housing of the adapter can be made easier if the housing of the adapter is rigid at least in some areas or is flexible and/or elastic at least in some areas. It is thus possible to widen the housing of the adapter for insertion of the electromedical device into the recess of the adapter and insert the implantable electromedical device into the recess. On account of the flexibility or elasticity of the housing, the housing can then press tightly onto the implantable electromedical device inserted into the recess and thereby contribute to the implantable electromedical device being safely secured on the housing of the adapter. Moreover, an adapter of this configuration, particularly if its recess has a corresponding size, can be designed to engage behind the implantable electromedical device in and/or counter to an attachment direction of the adapter for producing an electrical connection between the adapter and the implantable electromedical device and in this way engage around the latter with a form fit.

At least one wall of the housing delimiting the recess can expediently have at least one securing opening extending through the wall and/or a securing bore extending through the wall, through which at least one securing element, in particular a screw and/or a pin, is insertable and/or inserted for force-fit and/or form-fit engagement of the implantable electromedical device inserted into the recess in the position of use. The at least one securing element can cross right through the recess in the position of use. The at least one securing element can expediently engage in suture holes which are customarily present on an implantable electromedical device and which serve for sewing the electromedical device in the implanted position of use into the tissue of the tissue pocket and thereby fixing it in its position, so as to be able to secure the device in the recess of the housing of the adapter.

The housing of the adapter can be formed in one piece or from at least two housing parts, wherein the at least two housing parts are connectable and/or are connected to each other preferably releasably.

At least one wall or the wall delimiting the recess can be the one housing part and be connectable and/or connected releasably to the at least one other housing part and can be removed from the at least one other housing part for insertion of the implantable electromedical device into the recess.

To ensure that an electrical connection between the adapter and the electromedical device to be inserted into the recess of the adapter can be produced as easily as possible even under confined conditions, it may be advantageous if the first electrical terminal has at least one but in particular two preferably flexible contact pins which, in the position of use, fit into and can be pushed into a respective socket formed on the implantable electromedical device.

In a particularly advantageous embodiment of the invention, provision can additionally be made that the adapter located on the implantable electromedical device in the position of use engages behind the implantable electromedical device, specifically in and/or counter to an attachment direction of the adapter to the implantable electromedical device in order to produce an electrical connection between the adapter and the implantable electromedical device. It is thus possible that the adapter engages with a form fit around the implantable electromedical device at least in and/or counter to the attachment direction and impedes or even prohibits removal of the adapter from the implantable electromedical device in and/or counter to the attachment direction.

If the adapter has at least one contact pin, provision can be made that the adapter located on the implantable electromedical device in the position of use engages behind the implantable electromedical device, specifically in and/or counter to an insertion direction of at least one contact pin, for example the aforementioned contact pin, of the first electrical terminal of the adapter into one socket, for example the aforementioned socket, formed on the implantable electromedical device. In this way too it is possible to ensure that the adapter engages with a form fit around the implantable electromedical device at least in and/or counter to the insertion direction and impedes or even prohibits removal or withdrawal of the adapter from the implantable electromedical device.

Both embodiments of the invention serve directly to produce a stable and secure connection between the adapter and the implantable electromedical device.

This design of the adapter is advantageous since, in this way, relative movements between the adapter and the implantable electromedical device can be reduced still more reliably or can even be avoided. These relative movements can exert a pump action on body fluids which surround the adapter and the electromedical device in the implanted position of use and whose entry at a contact point between the adapter and the implantable electromedical device can lead to malfunctions and is therefore to be avoided

In this way, it is moreover possible that the adapter engages around the implantable electromedical device across a large surface area and, as has already been mentioned before, also with a form fit, and therefore the above-described sealing action of at least one wall of the housing can be supported. Moreover, with such a design of the adapter, inadvertent disconnection of the electrical connection between the adapter and the electromedical device can be made difficult or even prohibited.

The housing of the adapter may expediently have at least one externally accessible securing hole open at both ends and/or an externally accessible securing eyelet for suturing and/or anchoring the adapter, in its implanted position of use, in a tissue of the body.

Moreover, in order to transmit bipolar and/or quadripolar and/or multi-polar electromedical signals, in particular bipolar and/or quadripolar and/or multi-polar electromethcal stimulation impulses, from an implantable electromedical device to an electromedical signal transmitter, the adapter can be designed such that its first electrical terminal and its second electrical terminal each have two or four or more electrical contacts, in particular a number corresponding to the number of poles.

Electrical contacts of the first electrical terminal can be arranged on the or on at least one contact pin and/or on the one or more contact pins.

For medical reasons, it is expedient if the adapter is made of material suitable for a permanent implant, in particular of plastic and/or of silicone and/or of metal.

The invention also relates to an adapter with an implantable electromedical signal transmitter connected to the adapter in the position of use, wherein the electromedical signal transmitter is an implantable electromedical stimulation electrode and/or an implantable extension that is attachable to an implantable electromedical stimulation electrode. However, it is also possible that the implantable electromedical signal transmitter is an implantable electromedical sensing electrode, with which electromedical signals can be transmitted from or out of a body to an implantable electromedical device.

The invention also relates to an adapter with an implantable electromedical device attached to the adapter in the position of use, wherein the device is preferably an implantable cardiac pacemaker and/or an implantable neurostimulator and/or an implantable electromedical signal receiver and, in the position of use, is inserted into a recess or the recess of the adapter.

It will be noted that a unit is thus also claimed formed of an adapter together with an implantable electromedical device insertable or inserted into the recess of the adapter and attachable or attached to the first electrical terminal of the adapter, and with an implantable electromedical signal transmitter attachable or attached to the second electrical terminal.

It will also be noted that the expression “implantable electromedical device” comprises all customary devices of this kind and in particular implantable cardiac pacemakers, implantable neurostimulators and implantable electromedical signal receivers.

The expression “implantable electromedical signal transmitter” comprises all customary signal transmitters of this kind and in particular implantable electromethcal impulse pickups, for example stimulation electrodes and/or extensions for attachment to implantable electromedical signal transmitters, which receive stimulation impulses emitted by the implantable electromedical device and forward these signals to the body. However, the expression “implantable electromedical signal transmitter” also comprises so-called sensing electrodes, with which electromedical signals can be transmitted from a body, in particular from an organ of a body, to an electromedical signal receiver, in order to be recorded by the latter and to be stored for subsequent evaluation. Alternatively or in addition, the electromedical signal receiver can also transmit the signals via a suitable (e.g. wireless) interface to the environment (in the body or outside the body).

It will be appreciated that the subject matter of the invention is suitable for use both in the human body and also in the animal body.

### BRIEF DESCRIPTION OF TH DRAWINGS

Illustrative embodiments of the invention are explained in more detail below and depicted in sometimes highly schematic form in the drawing, in which:

FIG. 1 shows a first perspective view of an adapter according to the invention composed of several housing parts, with an implantable electromedical device inserted into the recess of the adapter,

FIG. 2 shows the multi-part adapter depicted in FIG. 1, with the implantable electromedical device depicted in FIG. 1, wherein the adapter is shown in an exploded view, and a wall of the housing delimiting the recess of the adapter can be seen, and also two securing pins serving as securing elements or securing means, and two contact pins of the first electrical terminal in the interior of the recess of the housing of the adapter,

FIG. 3 shows a perspective view of a further adapter according to the invention, with an electromedical device inserted into the recess of the adapter, wherein this adapter is designed in one piece,

FIG. 4 shows the adapter depicted in FIG. 3, with the electromedical device depicted likewise in FIG. 3, wherein the adapter is removed from the electromedical device, and two flexible and bendable contact pins of the first electrical terminal of the adapter, arranged in the interior of the recess of the adapter, and two securing pins can be seen which, in the position of use, can be inserted through two externally accessible securing openings, extending through a wall of the housing of the adapter, in order to fix the electromedical device, in its position of use, in the recess of the adapter,

FIG. 5 shows a side view of an adapter according to the invention on an implantable electromedical device designed as a neurostimulator, with an extension attached to the second electrical terminal of the adapter according to the invention and serving as a signal transmitter, and with a stimulation electrode attached to a distal end of the extension and serving as a further signal transmitter, and

FIG. 6 shows a side view of an adapter according to the invention mounted on an implantable electromedical device designed as a cardiac pacemaker, with a stimulation electrode attached to the second electrical terminal of the adapter and serving as signal transmitter.

### DETAILED DESCRIPTION OF THE PREFERRED EMBODIMENTS

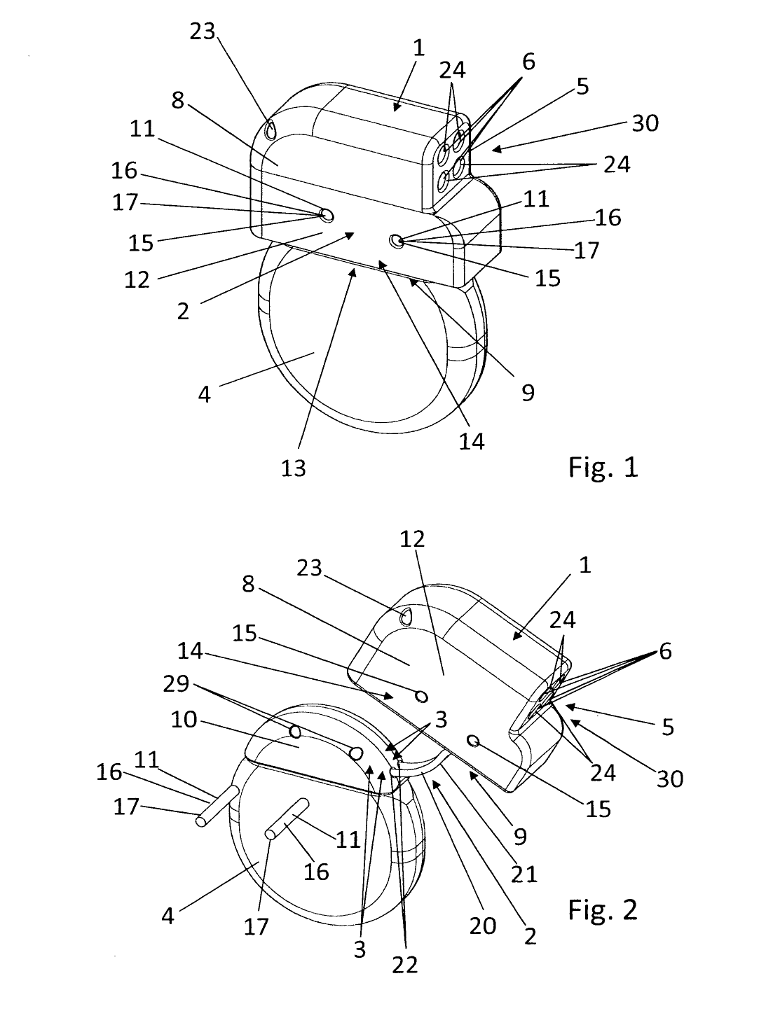

According to FIGS. 1 to 6, an adapter implantable in a body, and designated overall by reference number 1, has a first electrical terminal 2 with at least one electrical contact 3 for attaching an implantable electromedical device 4, and a second electrical terminal 5 with at least one electrical contact 6 for attaching an implantable electromedical signal transmitter 7 which, in the implanted position of use, is designed to transmit electromedical signals between the implantable electromedical device 4 and the body, in particular an organ of the body. The first electrical terminal 2 is electrically connected to the second electrical terminal 5 of the adapter 1 for the transmission of these electromedical signals.

Depending on the particular use, the implantable electromedical device 4 shown in FIGS. 1 to 4 can be, for example, a cardiac pacemaker, a neurostimulator and/or an electromedical signal receiver.

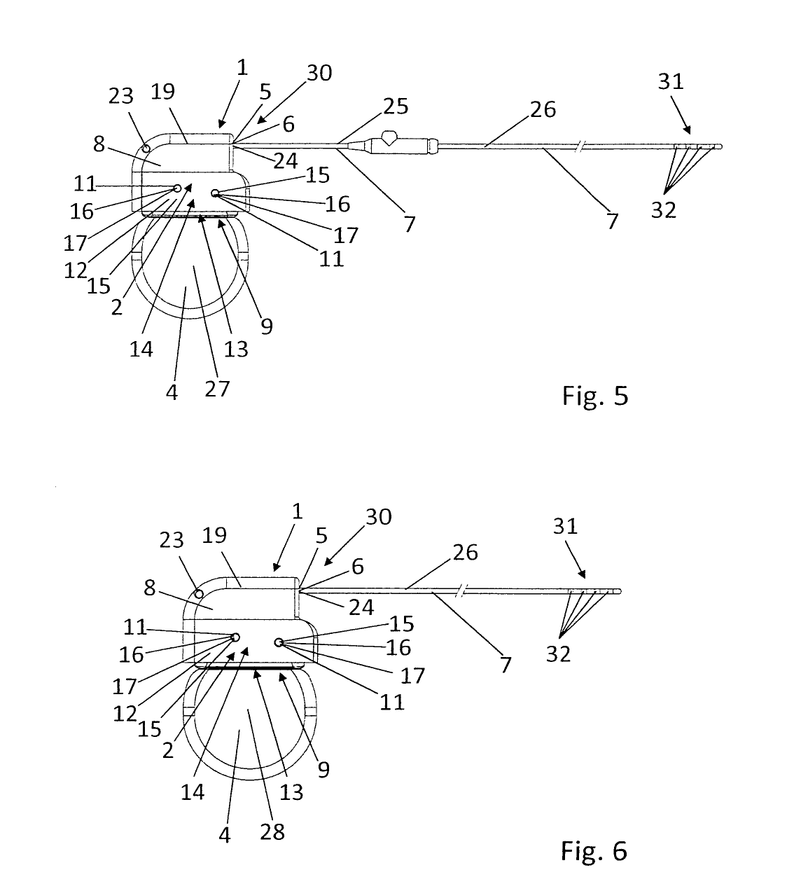

The implantable electromedical device 4 shown in FIG. 5 is a neurostimulator 27, while FIG. 6 shows an implantable electromedical device 4 in the form of a cardiac pacemaker 28.

The body can be a human body or also an animal body. A use of the adapter 1 in humans and animals is possible.

Electromedical signals are to be understood both as electromedical signals of the kind that are sent from the body through the signal transmitter to an implantable electromedical device 4, for example ECG or EEG signals, and also as stimulation and/or defibrillation impulses which are sent from an implantable electromedical device into and/or onto the body or an organ of the body.

The adapter 1 has a housing 8 on which the first electrical terminal 2 and the second electrical terminal 5 are formed. Moreover, the housing 8 has a recess 9 which is designed to receive and engage around at least part of the implantable electromedical device 4.

In the illustrative embodiments shown in the figures, the adapter 1 engages around a header or head part 10 of the device 4, and electromedical signal transmitters 7 directly fitting the device 4 can also be attached directly to the head part 10.

Depending on the particular use, these suitable signal transmitters 7 are, for example, electromedical impulse pickups, such as stimulation electrodes, or extensions to which stimulation electrodes can be attached. However, so-called sensing electrodes, with which electromedical signals emitted from organs can be transmitted to a suitable electromedical device 4, for example to a preferably implantable electromedical signal receiver, which then records the electromedical signals, can also be attached to the adapter 1 and used with the latter.

The adapter 1 has at least one securing means 11, in particular for releasable and/or form-fit and/or force-fit engagement of the impulse transmitter 4 inserted into the recess 9. FIGS. 2 and 4 in particular show that the first electrical terminal 2 for the implantable electromedical device 4 is formed in the interior of the recess 9 of the housing 8, and the second electrical terminal 5 for the implantable electrometrical signal transmitter 7 is formed on an externally accessible side or outer face 30 of the housing 8.

At least one wall 12 of the housing 8 delimiting the recess 9 is in this case designed for leak-tight closure of a gap 13 present between the wall 12 and the device 4 inserted into the recess 9. For this purpose, the wall 12 has a means for preferably leak-tight closure in the form of a seal or a sealing lip 14.

In the embodiment of the adapter 1 shown in FIGS. 1 and 2, the housing 8 of the adapter 1 is rigid in some areas and flexible or elastic in some areas, so as to be able to insert the device 4, in its position of use shown in FIG. 1, into the recess 9 of the housing 8 of the adapter 1.

All of the figures show that at least one wall of the housing 8 delimiting the recess, here the wall 12, has two securing openings or securing bores 15 extending through the wall 12. To ensure that the electromedical device 4 inserted into the recess 9 is fixed with force-fit and/or form-fit engagement, two securing elements 16, here two pins 17, are insertable or are inserted as securing means 11 through the two securing openings or securing bores 15, wherein the two securing elements 16 or pins 17 cross through the recess 9 in the inserted position of use.

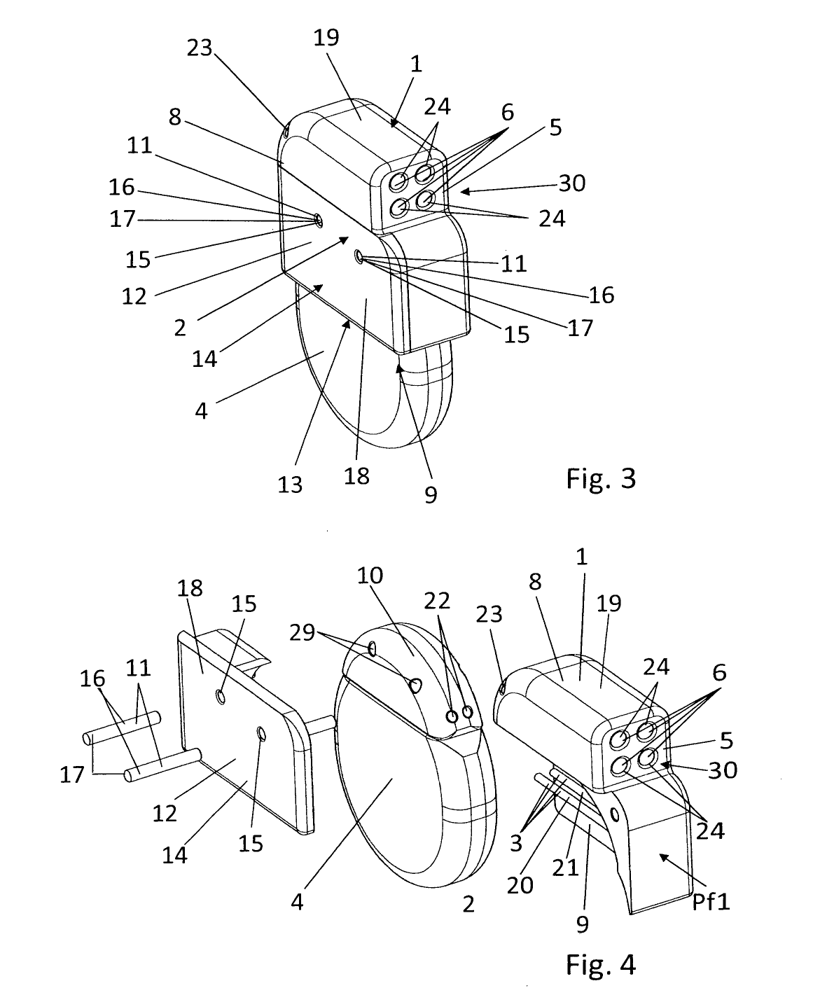

To secure the implantable electromedical device 4 in the recess 9, the two pins 17 are each inserted respectively into one of two suture holes 29 present in the head part 10 of the device 4 and thus reach their securing positions shown in FIGS. 1 and 3. When the device 4 is used without the adapter 1, a securing suture is guided through the two suture holes 29 of the impulse transmitter 4 in order to anchor the device 4 in the tissue of a patient.

Inserted in the securing position, the pins 17 are connected to the housing 8 of the adapter 1 preferably releasably and in a manner known per se, in particular by adhesive bonding, welding, clamping or pressing.

The housing 8 of the adapter shown in FIGS. 1 and 2 is designed in one piece.

The housing 8 of the adapter shown in FIGS. 3 and 4 is composed of two housing parts 18 and 19, which are releasably connectable to each other and, according to FIG. 3, are releasably connected to each other.

The wall 12 delimiting the recess 9 forms the one housing part 18, which is releasably connectable to the other housing part 19 and, in the position of use, is so connected. To insert the implantable electromedical device 4 into the recess 9, the wall 12 delimiting the recess 9, i.e. the one housing part 18, is detachable from the other housing part 19.

According to FIGS. 2 and 4, the first electrical terminal 2 has two contact pins 20 and 21 which, in the position of use, each fit in and can be pushed into a respective socket 22 formed on the electromedical device 4.

In the adapter 1 shown in FIGS. 1 and 2 and having the one-piece housing 8, the two contact pins 20 and 21 are flexible in order to facilitate attachment of the electromedical device 4, whereas, in the adapter 1 shown in FIGS. 3 and 4 and having the multi-part housing 8, the two contact pins 20 and 21 are rigid, such that, for attachment to the device 4, the adapter 1 is pushed from one side in arrow direction Pf1 onto the device 4, wherein the two rigid contact pins 20 and 21 are inserted into sockets 22 formed on the implantable electromedical device 4, and electrical contact is thus established between the adapter 1 and the device 4.

All of the figures show that the housing 8 of the adapter 1 an externally accessible securing hole 23 open at both ends for suturing and/or anchoring the adapter 1 in its implanted position of use in the tissue of a tissue pocket of a patient. Through this securing hole 23, which is designed as a securing channel open at both ends and reaching through the housing 8 of the adapter 1, it is possible to guide a suture with which the adapter 1 is fixed in its implanted position of use, and slipping of the adapter 1 is prevented.

In an embodiment of the adapter 1 not shown in the figures, the housing 8 of the adapter 1 has at least one externally accessible securing eyelet for suturing and/or anchoring the adapter 1 in its implanted position of use in a tissue pocket.

Both adapters 1 shown in FIGS. 1 to 6 are designed, in order to transmit quadripolar electromedical signals, in particular to transmit stimulation impulses, from the implantable electromedical device 4 to an electromedical signal transmitter 7, such that the first electrical terminal 2 and the second electrical terminal 5 each have four electrical contacts 3 and 6, i.e. a number corresponding to the number of poles.

In the illustrative embodiments of the adapter 1 shown in the figures, the total four electrical contacts 3 of the first electrical terminal 2 are arranged on the two contact pins 20 and 21. Each of the two contact pins 20 and 21 has two of the total of four electrical contacts 3, while one of the total of four electrical contacts 6 of the second electrical terminal 5 of the adapter 1 is in each case arranged or formed respectively in the interior of a connection socket 24. The second electrical terminal 5 thus has a total of four individual connection sockets 24, i.e. a number corresponding to the number of poles, for attachment of the signal transmitter 4.

All of the figures illustrate that the adapter 1 located in its position of use engages behind the implantable electromedical device 4 in and/or counter to an attachment direction of the adapter 1 to the implantable electromedical device 4 in order to produce an electrical connection between the adapter 1 and the implantable electromedical device 4. In the illustrative embodiments shown in the figures, this signifies specifically that the adapter 1 located in its position of use engages behind the implantable electromedical device 4 in and counter to an insertion direction of the two contact pins 20, 21 of the first electrical terminal 2 of the adapter 1 into the socket 22 formed on the implantable electromedical device 4. In this way, a form-fit engagement is generated between the adapter 1 and the implantable electromedical device 4, at least in and counter to the insertion direction of the contact pins 20, 21, such that the adapter 1 encloses the implantable electromedical device 4 across a large surface area and is reliably fixed thereon.

All of the adapters 1 shown in the figures are comprised of material suitable for a permanent implant, for example plastic, silicone or metal.

According to FIG. 5, the electromedical signal transmitter 7 attached to the adapter 1 in the position of use is an extension 25 that can be attached to an electronic stimulation electrode and, according to FIG. 6, it is an electromedical stimulation electrode 26.

In an illustrative embodiment of the invention not shown here, the signal transmitter 7 is designed as a sensing electrode, with which signals sent from organs can be transmitted to an electromedical device 4, in particular to an electromedical signal receiver, and can be recorded by the latter for subsequent evaluation.

The signal transmitter 7 can also be designed as a combined form of electromedical stimulation electrode and/or extension and/or sensing electrode, i.e. can have a dual function or even a triple function.

An extension 25 as shown in FIG. 5 is used in particular when the output site of the electromedical signals and the site where the electromedical signals are received are so far from each other that, in order to bridge the distance between the output site of the electromedical signals and the site where they are received, whether from the device 4 to the body or vice versa, the use of an extension 25 is expedient or necessary. Electromedical signals issuing from the electromedical device 4 or the body stimulation signals to the body and the implant position of the adapter 1 with the electromedical device 4 lie so far apart that, in order to bridge the distance between implant position and the output site of the electromedical signals, whether from the device 4 to the body or vice versa, the use of an extension 25 is expedient or necessary.

The extension 25 is used especially in neurostimulators, which are sometimes also designated as brain pacemakers. Neurostimulators can usually be implanted beneath a patient's clavicle. However, the stimulation impulses of the neurostimulator are intended to be output in the patient's brain via a stimulation electrode, i.e. not in direct proximity to the location where it is implanted. The electromedical signals that are output from the electromedical device 4 shown in FIG. 5 and designed as a neurostimulator 27 are stimulation impulses for a patient's brain and, by way of an extension 25 connected to the adapter 1 and running through the neck into the patient's head from the tissue pocket created for receiving the adapter 1 and the electromedical device 4, they are transmitted to a stimulation electrode 26 which is located in the patient's head in the position of use and which is connected to the patient's brain.

As FIG. 5 shows, the stimulation electrode 26, at its distal end 31 contacting the patient's brain in the position of use, has four individual poles or contacts 32 for outputting stimulation impulses to the brain.

The electromedical device 4 shown in FIG. 6 is a cardiac pacemaker 28, and a stimulation electrode 26 as an electromedical signal transmitter 7 is attached to the second electrical terminal 5 of the pacemaker 8 without an interpositioned extension 25. The stimulation electrode 26 shown in FIG. 6 has, at its distal end 31 contacting the patient's heart in the position of use, four individual poles or contacts 32 for outputting stimulation impulses to the heart.

The adapter 1, implantable in a human body, has the first electrical terminal 2 with at least one electrical contact 3 for attaching the implantable electromedical device 4, and the second electrical terminal 5 with at least one electrical contact 6 for attaching the implantable electromedical signal transmitter 7, in particular the stimulation electrode 26 or the extension 25. The first electrical terminal 2 and the second electrical terminal 5 of the adapter 1 are electrically connected to each other in order to transmit electrical stimulation impulses. The adapter 1 has the housing 8, on which the first electrical terminal 2 and the second electrical terminal 5 are formed. Moreover, the housing 8 has the recess 9, which is designed to receive and engage around at least part of the implantable electromedical device 4.

1. A medical adapter (1) implantable in a body, comprising a first electrical terminal (2) with at least one electrical contact (3) for attaching an implantable electromedical device (4), a second electrical terminal (5) with at least one second electrical contact (6) for attaching an implantable electromedical signal transmitter (7) which, in an implanted position of use, is designed to transmit electromedical signals between the body and the implantable electromedical device (4), wherein the first electrical terminal (2) and the second electrical terminal (5) of the adapter (1) are electrically connected to each other in order to transmit electromedical signals, the adapter (1) has a housing (8), on which the first electrical terminal (2) and the second electrical terminal (5) are formed, and the housing (8) has a recess (9), which is designed to receive and engage around at least part of the implantable electromedical device (4).

2. The adapter as claimed in claim 1, wherein the adapter (1) has at least one securing element (11) for engagement of the device (4) inserted into the recess (9).

3. The adapter as claimed in claim 1, wherein the first electrical terminal (2) for the implantable electromedical device (4) is formed in an interior of the recess (9) of the housing (8), and the second electrical terminal (5) for the implantable electromedical signal transmitter (7) is formed on an externally accessible side or outer face (30) of the housing (8).

4. The adapter as claimed in claim 1, wherein at least one wall (12) of the housing (8) delimiting the recess (9) is designed for closure of a gap (13) present between the wall (12) and the electromedical device (4) inserted into the recess (9).

5. The adapter as claimed in claim 1, wherein the housing (8) of the adapter (1) is rigid at least in some areas or is flexible at least in some areas, or both.

6. The adapter as claimed in claim 1, wherein at least one wall (12) of the housing (8) delimiting the recess has at least one securing opening or securing bore (15) extending through the wall (12), through which at least one securing element (16) is at least one of insertable or inserted for engagement of the implantable electromedical device (4) inserted into the recess (9), and the at least one securing element (16) crosses right through the recess (9) in the position of use.

7. The adapter (1) as claimed in claim 1, wherein the housing (8) is formed in one piece or from at least two housing parts (18, 19), with the at least two housing parts (18, 19) being connectable to each other in the position of use.

8. The adapter as claimed in claim 1, wherein the housing is formed from at least two housing parts and at least one wall (12) delimiting the recess (9) is on one of the housing parts (18) and is connectable to the at least one other housing part (19) and is removable from the at least one other housing part (19) for insertion of the implantable electromedical device (4) into the recess (9).

9. The adapter as claimed in claim 1, wherein the first electrical terminal (2) has at least one, flexible contact pins (20, 21) which, in a position of use, fits into and is pushable into a respective socket (22) formed on the implantable electromedical device (4).

10. The adapter as claimed in claim 1, wherein the adapter (1) located on the implantable electromedical device (4) in the position of use engages behind the implantable electromedical device (4) in or counter to an attachment direction of the adapter (1) to the implantable electromedical device (4) in order to produce an electrical connection between the adapter (1) and the implantable electromedical device (4), such that the adapter (1), in the position of use, engages with a form fit around the implantable electromedical device (4) in or counter to an attachment direction, such that the adapter (1) located on the implantable electromedical device (4) at least one of in the position of use engages behind the implantable electromedical device (4) in or counter to an insertion direction of or the at least one contact pin (20, 21) of the first electrical terminal (2) of the adapter (1) into one socket formed on the implantable electromedical device (4), such that the adapter (1), in the position of use, engages around the implantable electromedical device (4) with a form fit at least one of in or counter to a direction of the insertion direction.

11. The adapter as claimed in claim 1, wherein the housing (8) of the adapter (1) has at least one externally accessible securing hole (23) open at both ends or an externally accessible securing eyelet for at least one of suturing or anchoring the adapter (1), in its implanted position of use, in a tissue of the body.

12. The adapter as claimed in claim 1, wherein in order to transmit at least one of bipolar or quadripolar or multi-polar electromedical signals from the implantable electromedical device (4) to an electromedical signal transmitter, the adapter (1) is designed such that the first electrical terminal (2) and the second electrical terminal (5) each have two or four or more electrical contacts (3, 6), in particular a number corresponding to a number of the poles.

13. The adapter as claimed in claim 1, wherein the electrical contacts (3) of the first electrical terminal (2) are arranged on at least one contact pin.

14. The adapter as claimed in claim 1, wherein the adapter (1) is made of material suitable for a permanent implant.

15. The adapter as claimed in claim 1, wherein with an implantable electromedical signal transmitter (7) attached to the adapter (1) in the position of use, wherein the electromedical signal transmitter (7) comprises at least one of an electromedical stimulation electrode (26), an extension (25) attachable to an electromedical stimulation electrode (26), or a sensing electrode.

16. The adapter as claimed in claim 1, wherein the device (4) is at least one of a cardiac pacemaker (28), a neurostimulator (27), or an electromedical signal receiver and, in the position of use, is inserted into the recess (9) of the adapter (1).
